(19) United States
(12) Patent Application Publication
(10) Pub. No.: US 2010/0292444 A1
(43) Pub. Date: Nov. 18, 2010

(54) Novel Prolactin Compounds

(75) Inventors: Jens Breinholt, Dyssegard (DK); Per Franklin Nielsen, Malov (DK); Leif Christensen, Roskilde (DK); Leif Nørskov-Lauritsen, Tappernoje (DK); Anders Swensson, Malmo (SE)

Correspondence Address: NOVO NORDISK, INC.;INTELLECTUAL PROPERTY DEPARTMENT, 100 COLLEGE ROAD WEST, PRINCETON, NJ, 08540, US

(73) Assignee: Novo Nordisk A/S, Bagsvaerd (DK)

(21) Appl. No.: 12/666,395

(22) PCT Filed: Jul. 3, 2008
(86) PCT No.: PCT/EP2008/058593

§ 371 Date: May 12, 2010

(30) Foreign Application Priority Data
Jul. 5, 2007 (EP) 07111799.8

(51) Int. Cl. C07K 14/435; C07H 21/04; C12N 15/63; C12N 5/00; C07K 16/00

(52) U.S. Cl. 530/399; 536/23.1; 435/320.1; 435/325; 530/387.3

(57) ABSTRACT

The present invention relates to prolactin analogues, which analogues have increased resistance to deamidation.

### FIELD OF THE INVENTION

The invention relates to novel prolactin compounds, to methods of preparing said compounds, to pharmaceutical compositions comprising these compounds and to the use of the compounds for the treatment of diseases.

### BACKGROUND OF THE INVENTION

A major issue in the development of protein drugs is the stability of the protein from its production to its final administration, and major efforts are typically put in to identification of a formulation that provides adequate physical and chemical stability of the drug substance.

Chemical instability of proteins is mainly associated with oxidative or hydrolytic modifications at sensitive residues in the amino acid sequence. For example, the side chain amide group in glutaminyl or asparaginyl residues may be hydrolysed to form a free carboxylic acid in a process called deamidation. This can lead to the formation of chemical degradation products with altered and potentially undesired biological properties.

The rate of deamidation is strongly dependent on several physical and chemical conditions, particularly temperature, pH, dielectric constant of solvent but also the presence of certain ions, such as phosphate which has been shown to catalyze the Asn deamidation reactions. Furthermore, both the primary amino acid sequence and secondary structure strongly influence the rate of deamidation (Robinson, N. E., Robinson, Z. W., Robinson, B. R., Robinson, A. L., Robinson, J. A., Robinson, M. L., and Robinson, A. B. (2004) Journal of Peptide Research 63, 426-436). In the primary sequence particularly the nature of the amino acid immediately after (position n+1) influences the deamidation rate of an Asn-residue (position n). Asn-residues followed in the amino acid sequence by a Glycine are particularly susceptible to deamidation. The rate of deamidation for an Asn-residue (position n) is to a lesser extent influenced by the nature of amino acid in position n−1. Asn residues situated in loosely structured and solvent exposed peptide segments are typically more susceptible to deamidation as compared to Asn-residues situated in secondary structure elements (helix and sheet).

PRL has been reported to readily undergo deamidation, and deamidated PRL-variants have been encountered in most species examined, including mouse, rat, sheep and human (Sinha, Y. N. (1995) Endocrine Reviews 16 354-369). Deamidation of PRL has been shown to alter its biological properties. Thus, reduced potency were reported for deamidated forms of rat and mouse PRL (Haro, L. S. and Talamantes, F. J. (1985) Endocrinology 116 353-358, Sinha, Y. N. and Gilligan, T. A. (1981) Endocrinology 108 1091-1094). Nyberg et al. (Nyberg, F., Roos, P., and Wide, L. (1980) Biochimica et Biophysica Acta 625 255-265) separated human pituitary PRL in to separate isoforms differing in their net charges and attributed to deamidated PRL-species. The deamidated isoforms of hPRL were shown to possess similar or even slightly increased lactogenic activity as measured by the pigeon crop-sac assay.

Deamidation of prolactin and prolactin analogues has been shown to represent a problem with respect to manufacturing, storage and biological activity. Thus, it is desirable to develop a prolactin analogue that exhibits improved stability with respect to deamidation, and which retains the desired biological activity.

US20060111293 relates to prolactin variants mutated in positions 41-57. US20060287508 relates to supression of deamidation of antibodies by substitution of a glycine located adjacent to an asparagine.

### SUMMARY OF THE INVENTION

The present invention is concerned with increasing the stability of prolactin or a prolactin analogue.

The present invention provides prolactin or a prolactin analogue having an amino acid mutation in the position corresponding to position 55, 56, or 57 of SEQ ID No. 1.

The present invention also provides a method for stabilising prolactin or a prolactin analogue comprising mutating one or more amino acids in positions resulting in induction of secondary structure at a position corresponding to position 55, 56, or 57 of SEQ ID No. 1.

### DESCRIPTION OF THE FIGURES

FIG. 1. Sequence of wtPRL with possible deamidation-sites (Asn-residues) highlighted.

FIG. 2. Ion exchange chromatography (MonoQ column). Peaks 0, 1, 2, 3 and 4 elute at 9.0, 10.4, 11.5, 12.4 and 12.8, respectively (see Example 1).

FIG. 3. Amino acid sequence of human prolactin (SEQ ID No. 1)

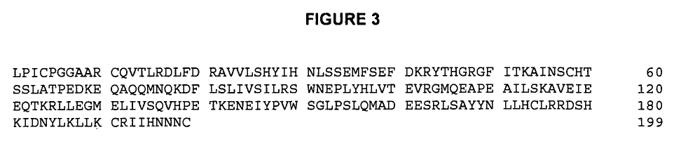

### DESCRIPTION OF THE INVENTION

Using recombinantly produced hPRL and hPRL-variants we have experimentally confirmed that hPRL readily undergoes deamidation, particularly under alkaline conditions. Furthermore, we have demonstrated that the primary site of deamidation is Asn-56 of SEQ ID No. 1. Surprisingly, we observe that PRL and PRL-analogues deamidated at Asn-56 possess a markedly reduced affinity for the prolactin receptor.

Prolactin (PRL) is a single chain polypeptide of 199 amino acid residues with a molecular weight of about 24,000 Daltons. It is synthesised in the adenohypophysis (anterior pituitary gland), in the breast and in the decidua and has a structure similar to that of growth hormone (GH) and placental lactogen (PL). The molecule is folded due to the activity of three disulfide bonds. The sequence of human prolactin is given in SEQ ID No. 1.

Human prolactin (hPRL) has two separate and different binding sites (site 1 and site 2) that each interact with a prolactin receptor to form a 1:2 ligand-receptor complex. The classical model (Fuh, G., Cunningham, B. C., Fukunaga, R., Nagata, S., Goeddel, D. V., and Wells, J. A. (1992) Science 256 1677-1680) for activation of the PRL and GH receptors (PRLR and GHR, respectively) describes the signaling molecular entity as a ternary complex between one hormone molecule and a receptor homo-dimer assembled in a strictly sequential and hormone dependent fashion: first the hormone ligand engages via site 1 in high affinity binding to one receptor chain forming a 1:1 hormone/receptor complex, which constitutes the template for binding a second, identical receptor molecule resulting in the active 1:2 complex. However, this model has been challenged by an increasing body of experimental evidence. Initially reported for the homologous human erythropoietin receptor (hEPOR) (Constantinescu, S. N., Keren, T., Socolovsky, M., Nam, H. S., Henis, Y. I., and Lodish, H. F. (2001) Proceedings of the National Academy of Sciences of the United States of America 98 4379-4384) and later for hGHR (Brown, R. J., Adams, J. J., Pelekanos, R. A., Wan, Y., McKinstry, W. J., Palethorpe, K., Seeber, R. M., Monks, T. A., Eidne, K. A., Parker, M. W., and Waters, M. J. (2005) Nature Structural & Molecular Biology 12 814-821) and hPRLR (Qazi, A. M., Tsai-Morris, C. H., and Dufau, M. L. (2006) Molecular Endocrinology 20 1912-1923), these studies support that preformed, inactive dimers exist in the absence of hormone. This infers that receptor dimerization is a necessary, but not sufficient, event for receptor activation, and, notably, not a strictly ligand dependent one. For both hEPOR (Seubert, N., Royer, Y., Staerk, J., Kubatzky, K. F., Moucadel, V., Krishnakumar, S., Smith, S. O., and Constantinescu, S. N. (2003) Molecular Cell 12 1239-1250) and hGHR (Brown, R. J., Adams, J. J., Pelekanos, R. A., Wan, Y., McKinstry, W. J., Palethorpe, K., Seeber, R. M., Monks, T. A., Eidne, K. A., Parker, M. W., and Waters, M. J. (2005) Nature Structural & Molecular Biology 12 814-821) models have been proposed, where receptor activation involves relative rotations and movements of receptor subunits induced by hormone binding. Allosteric reorganization of the intracellular receptor domains brings about activation of the receptor associated Janus family of tyrosine kinases JAK2 or JAK1, which stimulate signal transducers and activators of transcription STAT5 or STAT3, respectively. Receptor activation also leads to the activation of Ras/Raf/MAPK kinase/Erk and phosphatidylinositol 3-kinase/Akt signalling pathways. It is primarily via these pathways that the receptors for these ligands induce cell differentiation, proliferation, and/or survival (lhle, J. N. and Kerr, I. M. (1995) Trends in Genetics 11 69-74)

The term “protein”, “polypeptide” and “peptide” as used herein means a compound composed of at least five constituent amino acids connected by peptide bonds. The constituent amino acids may be from the group of the amino acids encoded by the genetic code and they may be natural amino acids which are not encoded by the genetic code, as well as synthetic amino acids. Natural amino acids which are not encoded by the genetic code are e.g. hydroxyproline, y-carboxyglutamate, ornithine, phosphoserine, D-alanine and D-glutamine. Synthetic amino acids comprise amino acids manufactured by chemical synthesis, i.e. D-isomers of the amino acids encoded by the genetic code such as D-alanine and D-leucine, Aib (a-aminoisobutyric acid), Abu (a-aminobutyric acid), Tle (tert-butylglycine), β-alanine, 3-aminomethyl benzoic acid and anthranilic acid.

The term “analogue” as used herein referring to a polypeptide means a modified peptide wherein one or more amino acid residues of the peptide have been substituted by other amino acid residues and/or wherein one or more amino acid residues have been deleted from the peptide and or wherein one or more amino acid residues have been added to the peptide. Such addition or deletion of amino acid residues can take place at any place in the amino acid sequence, for instance at the N-terminal of the peptide and/or at the C-terminal of the peptide and/or within the sequence. All amino acids for which the optical isomer is not stated are to be understood to mean the L-isomer.

The term “prolactin analogue” as used herein refers to an analogue of prolactin, which has the capability of binding to the prolactin receptor. A simple system is used to describe analogues of prolactin. For example, G129R-PRL designates an analogue of prolactin formally derived from prolactin by substituting the naturally occurring amino acid residue Glycine (G) in position 129 with Arginine (R). PRL(9-199) and PRL(12-199) designates an analogue formally derived from PRL by removal of the first 8 or 11 amino acids of the chain.

In one embodiment, the prolactin analogue has an amino acid sequence having at least 80% identity to SEQ ID No. 1. In one embodiment, the prolactin analogue has an amino acid sequence having at least 85%, such at least 90%, for instance at least 95%, such as for instance at least 99% identity to SEQ ID No. 1.

The term “identity” as known in the art, refers to a relationship between the sequences of two or more peptides, as determined by comparing the sequences. In the art, “identity” also means the degree of sequence relatedness between peptides, as determined by the number of matches between strings of two or more amino acid residues. “Identity” measures the percentage of identical matches between the smaller of two or more sequences with gap alignments (if any) addressed by a particular mathematical model or computer program (i.e., “algorithms”). Identity of related peptides can be readily calculated by known methods. Such methods include, but are not limited to, those described in Computational Molecular Biology, Lesk, A. M., ed., Oxford University Press, New York, 1988; Biocomputing: Informatics and Genome Projects, Smith, D. W., ed., Academic Press, New York, 1993; Computer Analysis of Sequence Data, Part 1, Griffin, A. M., and Griffin, H. G., eds., Humana Press, New Jersey, 1994; Sequence Analysis in Molecular Biology, von Heinje, G., Academic Press, 1987; Sequence Analysis Primer, Gribskov, M. and Devereux, J., eds., M. Stockton Press, New York, 1991; and Carillo et al., SIAM J. Applied Math. 48, 1073 (1988).

Preferred methods to determine identity are designed to give the largest match between the sequences tested. Methods to determine identity are described in publicly available computer programs. Preferred computer program methods to determine identity between two sequences include the GCG program package, including GAP (Devereux et al., Nucl. Acid. Res. 12, 387 (1984); Genetics Computer Group, University of Wisconsin, Madison, Wis.), BLASTP, BLASTN, and FASTA (Altschul et al., J. Mol. Biol. 215, 403-410 (1990)). The BLASTX program is publicly available from the National Center for Biotechnology Information (NCBI) and other sources (BLAST Manual, Altschul et al. NCB/NLM/NIH Bethesda, Md. 20894; Altschul et al., supra). The well known Smith Waterman algorithm may also be used to determine identity.

For example, using the computer algorithm GAP (Genetics Computer Group, University of Wisconsin, Madison, Wis.), two peptides for which the percentage sequence identity is to be determined are aligned for optimal matching of their respective amino acids (the “matched span”, as determined by the algorithm). A gap opening penalty (which is calculated as 3.times. the average diagonal; the “average diagonal” is the average of the diagonal of the comparison matrix being used; the “diagonal” is the score or number assigned to each perfect amino acid match by the particular comparison matrix) and a gap extension penalty (which is usually 1/10 times the gap opening penalty), as well as a comparison matrix such as PAM 250 or BLOSUM 62 are used in conjunction with the algorithm. A standard comparison matrix (see Dayhoff et al., Atlas of Protein Sequence and Structure, vol. 5, supp. 3 (1978) for the PAM 250 comparison matrix; Henikoff et al., Proc. Natl. Acad. Sci. USA 89, 10915-10919 (1992) for the BLOSUM 62 comparison matrix) is also used by the algorithm.

Preferred parameters for a peptide sequence comparison include the following: Algorithm: Needleman et al., J. Mol. Biol. 48, 443-453 (1970); Comparison matrix: BLOSUM 62 from Henikoff et al., PNAS USA 89, 10915-10919 (1992); Gap Penalty: 12, Gap Length Penalty: 4, Threshold of Similarity: 0.

The GAP program is useful with the above parameters. The aforementioned parameters are the default parameters for peptide comparisons (along with no penalty for end gaps) using the GAP algorithm.

In one embodiment, the prolactin analogue has an amino acid sequence, which sequence is at least 80% similar to SEQ ID No. 1. In one embodiment, the prolactin analogue has an amino acid sequence, which sequence is at least 85%, such as at least 90%, for instance at least 95%, such as for instance at least 99% similar to SEQ ID No. 1.

The term “similarity” is a concept related to identity, but in contrast to “identity”, refers to a sequence relationship that includes both identical matches and conservative substitution matches. If two polypeptide sequences have, for example, (fraction ( 10/20)) identical amino acids, and the remainder are all non-conservative substitutions, then the percentage identity and similarity would both be 50%. If, in the same example, there are 5 more positions where there are conservative substitutions, then the percentage identity remains 50%, but the percentage similarity would be 75% ((fraction ( 15/20))). Therefore, in cases where there are conservative substitutions, the degree of similarity between two polypeptides will be higher than the percentage identity between those two polypeptides.

Conservative modifications of a peptide comprising a given amino acid sequence (and the corresponding modifications to the encoding nucleic acids) will produce peptides having functional and chemical characteristics similar to those of a peptide comprising the given amino acid sequence. In contrast, substantial modifications in the functional and/or chemical characteristics of such peptide as compared to an original peptide may be accomplished by selecting substitutions in the amino acid sequence that differ significantly in their effect on maintaining (a) the structure of the molecular backbone in the area of the substitution, for example, as a sheet or helical conformation, (b) the charge or hydrophobicity of the molecule at the target site, or (c) the bulk of the side chain.

For example, a “conservative amino acid substitution” may involve a substitution of a native amino acid residue with a normative residue such that there is little or no effect on the polarity or charge of the amino acid residue at that position. Furthermore, any native residue in the polypeptide may also be substituted with alanine, as has been previously described for “alanine scanning mutagenesis” (see, for example, MacLennan et al., Acta Physiol. Scand. Suppl. 643, 55-67 (1998); Sasaki et al., Adv. Biophys. 35, 1-24 (1998), which discuss alanine scanning mutagenesis).

Desired amino acid substitutions (whether conservative or non-conservative) may be determined by those skilled in the art at the time such substitutions are desired. For example, amino acid substitutions can be used to identify important residues of the peptides according to the invention, or to increase or decrease the affinity of the peptides described herein for the receptor in addition to the already described mutations. Naturally occurring residues may be divided into classes based on common side chain properties:

1) hydrophobic: norleucine, Met, Ala, Val, Leu, Ile;

2) neutral hydrophilic: Cys, Ser, Thr, Asn, Gln;

3) acidic: Asp, Glu;

4) basic: His, Lys, Arg;

5) residues that influence chain orientation: Gly, Pro; and

6) aromatic: Trp, Tyr, Phe.

In making such changes, the hydropathic index of amino acids may be considered. Each amino acid has been assigned a hydropathic index on the basis of their hydrophobicity and charge characteristics, these are: isoleucine (+4.5); valine (+4.2); leucine (+3.8); phenylalanine (+2.8); cysteine/cystine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5).

The importance of the hydropathic amino acid index in conferring interactive biological function on a protein is understood in the art. Kyte et al., J. Mol. Biol. 157, 105-131 (1982). It is known that certain amino acids may be substituted for other amino acids having a similar hydropathic index or score and still retain a similar biological activity. In making changes based upon the hydropathic index, the substitution of amino acids whose hydropathic indices are within ±2 is preferred, those that are within ±1 are particularly preferred, and those within ±0.5 are even more particularly preferred.

The greatest local average hydrophilicity of a protein, as governed by the hydrophilicity of its adjacent amino acids, correlates with its immunogenicity and antigenicity, i.e., with a biological property of the protein.

The following hydrophilicity values have been assigned to amino acid residues: arginine (+3.0); lysine (+3.0); aspartate (+3.0±1); glutamate (+3.0±1); serine (+0.3); asparagine (+0.2); glutamine (+0.2); glycine (0); threonine (−0.4); proline (−0.5±1); alanine (−0.5); histidine (−0.5); cysteine (−1.0); methionine (−1.3); valine (−1.5); leucine (−1.8); isoleucine (−1.8); tyrosine (−2.3); phenylalanine (−2.5); tryptophan (−3.4). In making changes based upon similar hydrophilicity values, the substitution of amino acids whose hydrophilicity values are within ±2 is preferred, those that are within ±1 are particularly preferred, and those within ±0.5 are even more particularly preferred.

As used herein the term ‘prolactin analogue’ also includes all analogues that act as a prolactin receptor antagonist. The term “prolactin receptor antagonist” as used herein refers to a ligand having antagonistic activity at the prolactin receptor, causing it to act as an inhibitor of one or more cellular processes. Such prolactin antagonistic activity may be measured by Western blot analysis of the phosphorylation status of STATS as set out in Langenheim, J. F. et al, Mol Endocrinol, 2006; 20(39):661-674.

Without wishing to be bound by any one theory, it is believed that a prolactin receptor ligand, that comprises one or more mutations that affect the structural integrity of ‘Site 2’, will not trigger the receptor, and activate subsequent signal transduction, because it can not productively interact with the second receptor chain. Thus, such a ligand does not activate the receptor and instead acts as prolactin receptor antagonist. Six prolactin receptor antagonists are currently known in the literature (Goffin et al. Endocrine Rev. 2005, 26, 400-422):

(a) G120R/K-hGH, a variant of human growth hormone;

(b) G120R-hPL, a variant of human placental lactogen;

(c) G129R-hPRL a full-length variant of human prolactin;

(d) S179D-hPRL, a full-length variant of human prolactin;

(e) G129R-hPRL (10-199), a truncated variant of human prolactin; and

(f) G129R-hPRL (15-199), a truncated variant of human prolactin.

In one embodiment, the prolactin or prolactin analogue comprises a mutation N56X, wherein X is an amino acid residue which is resistant to deamidation. It will be appreciated that the mutation does not significantly impair the binding of the ligand to the receptor, e.g. the mutated prolactin or prolactin analogue may for instance have at least 25%, such as at least 50%, for instance at least 60% of the binding affinity for the prolactin receptor of the unmutated prolactin or prolactin analogue, but the precise ratio is not that important as long as the prolactin analogue can bind the prolactin receptor efficiently, even prolactin analogues with moderately reduced binding affinity, but which are resistant to deamidation, are of interest. As an example from the related erythropoietin (Epo) system, a hyperglycosylated analog of Epo, called novel erythropoiesis-stimulating protein (NESP), has a lower affinity than Epo for the Epo receptor but has greater in vivo activity and a longer serum half-life than Epo (Gross, A. W. and Lodish, H. F. Journal of Biological Chemistry 281, 2024-2032 (2006))

In a further embodiment, X is glycine.

In one embodiment, the prolactin or prolactin analogue comprises a mutation S57Y, wherein Y is an amino acid residue that suppresses deamidation of the amino acid residue in position 56 of SEQ ID No. 1. In one embodiment, Y is any of the natural amino acids encoded by the genetic code other than serine and glycine. In one embodiment, Y is selected from the amino acids valine, leucine, isoleucine, tryptophan, tyrosine, phenylalanine, proline and threonine.

In one embodiment, the prolactin or prolactin analogue comprises a mutation 155Z, wherein Z is an amino acid residue that suppresses deamidation of the amino acid residue in position 56 of SEQ ID No. 1. In one embodiment, Z is any of the natural amino acids encoded by the genetic code other than isoleucine. In one embodiment, Z is any of the amino acids comprising valine, leucine, tryptophan, tyrosine, phenylalanine, proline and threonine.

Without being bound by theory, it is believed that N56 of prolactin makes contact with the receptor, and thus that deamidation of N56 reduces affinity for the receptor. Thus, the mutations as hereinbefore defined help to maintain the activity of prolactin or a prolactin analogue by increasing the chemical stability of prolactin or a prolactin analogue in respect of deamidation.

This is important from a manufacturing point of view, because the production and purification processes will be complicated by the formation of deamidated products. Furthermore, stabilized proteins will increase the shelf life of the final product and improve overall economy. Finally, increased stability is also expected to be correlated with improved pharmacokinetic properties of the protein.

The present invention also provides a method for stabilising prolactin or a prolactin analogue, which method comprises mutating one or more amino acids in a position resulting in induction of an altered secondary structure at a position corresponding to positions 55, 56 or 57 of SEQ ID No. 1.

The present invention also provides a method for stabilising prolactin or a prolactin analogue, which method comprises mutating one or more amino acids in a position resulting in induction of an altered secondary structure at a position corresponding to positions 55 or 57 of SEQ ID No. 1.

In one embodiment, the method comprises mutating one or more amino acids in the segment corresponding to positions 47-57 of SEQ ID No. 1.

In one embodiment, the method comprises mutating one or more amino acids corresponding to positions 55, 56 or 57 of SEQ ID No. 1.

The present invention also provides a method for stabilising prolactin or a prolactin analogue comprising substituting asparagine in position 56 of SEQ ID No. 1 with an amino acid residue which is resistant to deamidation.

In a further embodiment, the method comprises substituting asparagine in position 56 of SEQ ID No. 1 with glycine.

In a further embodiment, the method comprises substituting asparagine in position 56 of SEQ ID No. 1 with glutamine.

The present invention also provides a method for stabilising prolactin or a prolactin analogue comprising substituting the amino acid residue in the position corresponding to position 55 (isoleucine in wtPRL) or the amino acid residue in the position corresponding to position 57 (serine in wtPRL) of SEQ ID No. 1 with an amino acid residue which suppresses deamidation of the amino acid residue in position 56 of SEQ ID No. 1. In one embodiment, the method comprises substituting the amino acid in position 55 of SEQ ID No. 1 with an amino acid residue which is any of the 20 amino acids other than glycine and isoleucine and/or substituting the amino acid in position 57 of SEQ ID No. 1 with an amino acid residue which is any of the 20 amino acids other than serine and glycine.

Peptides and pharmaceutical compositions according to the present invention may be used in the treatment of diseases treatable by administration of prolactin antagonists, such as breast cancer.

The term “treatment” and “treating” as used herein means the management and care of a patient for the purpose of combating a condition, such as a disease or a disorder. The term is intended to include the full spectrum of treatments for a given condition from which the patient is suffering, such as administration of the active compound to alleviate the symptoms or complications, to delay the progression of the disease, disorder or condition, to alleviate or relief the symptoms and complications, and/or to cure or eliminate the disease, disorder or condition as well as to prevent the condition, wherein prevention is to be understood as the management and care of a patient for the purpose of combating the disease, condition, or disorder and includes the administration of the active peptides to prevent the onset of the symptoms or complications. The patient to be treated is preferably a mammal, in particular a human being, but it may also include animals, such as dogs, cats, cows, sheep and pigs. It is to be understood, that therapeutic and prophylactic (preventive) regimes represent separate aspects of the present invention.

A “therapeutically effective amount” of a peptide as used herein means an amount sufficient to cure, alleviate or partially arrest the clinical manifestations of a given disease and its complications. An amount adequate to accomplish this is defined as “therapeutically effective amount”. Effective amounts for each purpose will depend on the type and severity of the disease or injury as well as the weight and general state of the subject. It will be understood that determining an appropriate dosage may be achieved using routine experimentation, by constructing a matrix of values and testing different points in the matrix, which is all within the ordinary skills of a trained physician or veterinary.

As used herein the term “nucleic acid construct” is intended to indicate any nucleic acid molecule of cDNA, genomic DNA, synthetic DNA or RNA origin. The term “construct” is intended to indicate a nucleic acid segment which may be single- or double-stranded, and which may be based on a complete or partial naturally occurring nucleotide sequence encoding a peptide of interest. The construct may optionally contain other nucleic acid segments.

A nucleic acid construct of the invention may suitably be of genomic or cDNA origin, for instance obtained by preparing a genomic or cDNA library and screening for DNA sequences coding for all or part of the peptide by hybridization using synthetic oligonucleotide probes in accordance with standard techniques (cf. J. Sambrook et al, 1989, Molecular Cloning, A Laboratory Manual, 2d edition, Cold Spring Harbor, N.Y.) and by introducing the relevant mutations as it is known in the art.

A nucleic acid construct of the invention may also be prepared synthetically by established standard methods, e.g. the phosphoamidite method described by Beaucage and Caruthers, Tetrahedron Letters 22, 1859-1869 (1981), or the method described by Matthes et al., EMBO Journal 3, 801-805 (1984). According to the phosphoamidite method, oligonucleotides are synthesized, e.g. in an automatic DNA synthesizer, purified, annealed, ligated and cloned in suitable vectors.

Furthermore, the nucleic acid construct may be of mixed synthetic and genomic, mixed synthetic and cDNA or mixed genomic and cDNA origin prepared by ligating fragments of synthetic, genomic or cDNA origin (as appropriate), the fragments corresponding to various parts of the entire nucleic acid construct, in accordance with standard techniques.

The nucleic acid construct may also be prepared by polymerase chain reaction using specific primers, for instance as described in U.S. Pat. No. 4,683,202 or Saiki et al., Science 239, 487-491 (1988).

In one embodiment, the nucleic acid construct of the invention is a DNA construct which term will be used exclusively in the following for convenience. The statements in the following may also read on other nucleic acid constructs of the invention with appropriate adaptions as it will be clear for a person skilled in the art.

In one embodiment, the present invention relates to a recombinant vector comprising a DNA construct of the invention. The recombinant vector into which the DNA construct of the invention is inserted may be any vector which may conveniently be subjected to recombinant DNA procedures, and the choice of vector will often depend on the host cell into which it is to be introduced. Thus, the vector may be an autonomously replicating vector, i.e. a vector which exists as an extrachromosomal entity, the replication of which is independent of chromosomal replication, e.g. a plasmid. Alternatively, the vector may be one which, when introduced into a host cell, is integrated into the host cell genome and replicated together with the chromosome(s) into which it has been integrated.

The vector may be an expression vector in which the DNA sequence encoding the peptide of the invention is operably linked to additional segments required for transcription of the DNA. In general, the expression vector is derived from plasmid or viral DNA, or may contain elements of both. The term, “operably linked” indicates that the segments are arranged so that they function in concert for their intended purposes, e.g. transcription initiates in a promoter and proceeds through the DNA sequence coding for the peptide.

The promoter may be any DNA sequence which shows transcriptional activity in the host cell of choice and may be derived from genes encoding proteins either homologous or heterologous to the host cell.

Examples of suitable promoters for use in yeast host cells include promoters from yeast glycolytic genes (Hitzeman et al., J. Biol. Chem. 255, 12073-12080 (1980); Alber and Kawasaki, J. Mol. Appl. Gen. 1, 419-434 (1982)) or alcohol dehydrogenase genes (Young et al., in Genetic Engineering of Microorganisms for Chemicals (Hollaender et al, eds.), Plenum Press, New York, 1982), or the TPI1 (U.S. Pat. No. 4,599,311) or ADH2-4-c (Russell et al., Nature 304, 652-654 (1983)) promoters.

Examples of suitable promoters for use in filamentous fungus host cells are, for instance, the ADH3 promoter (McKnight et al., The EMBO J. 4, 2093-2099 (1985)) or the tpiA promoter. Examples of other useful promoters are those derived from the gene encoding A. oryzae TAKA amylase, Rhizomucor miehei aspartic proteinase, A. niger neutral α-amylase, A. niger acid stable α-amylase, A. niger or A. awamori glucoamylase (gluA), Rhizomucor miehei lipase, A. oryzae alkaline protease, A. oryzae triose phosphate isomerase or A. nidulans acetamidase. In one embodiment, the promoter of a vector according to the invention is selected from the TAKA-amylase or the gluA promoters.

Examples of suitable promoters for use in bacterial host cells include the promoter of the Bacillus stearothermophilus maltogenic amylase gene, the Bacillus licheniformis alpha-amylase gene, the Bacillus amyloliquefaciens BAN amylase gene, the Bacillus subtilis alkaline protease gen, or the Bacillus pumilus xylosidase gene, or by the phage Lambda P_{R} or P_{L} promoters or the E. coli lac, trp or tac promoters.

The DNA sequence encoding the peptide of the invention may also, if necessary, be operably connected to a suitable terminator, such as the human growth hormone terminator (Palmiter et al., op. cit.) or (for fungal hosts) the TPI1 (Alber and Kawasaki, op. cit.) or ADH3 (McKnight et al., op. cit.) terminators. The vector may further comprise elements such as polyadenylation signals (e.g. from SV40 or the adenovirus 5 Elb region), transcriptional enhancer sequences (e.g. the SV40 enhancer) and translational enhancer sequences (e.g. the ones encoding adenovirus VA RNAs).

The recombinant vector of the invention may further comprise a DNA sequence enabling the vector to replicate in the host cell in question.

When the host cell is a yeast cell, suitable sequences enabling the vector to replicate are the yeast plasmid 2p replication genes REP 1-3 and origin of replication.

When the host cell is a bacterial cell, sequences enabling the vector to replicate are DNA polymerase III complex encoding genes and origin of replication.

The vector may also comprise a selectable marker, e.g. a gene the product of which complements a defect in the host cell, such as the gene coding for dihydrofolate reductase (DHFR) or the Schizosaccharomyces pombe TPI gene (described by P. R. Russell, Gene 40, 125-130 (1985)), or one which confers resistance to a drug, e.g. ampicillin, kanamycin, tetracyclin, chloramphenicol, neomycin, hygromycin or methotrexate. For filamentous fungi, selectable markers include amdS, pyrG, arqB, niaD and sC.

To direct a peptide of the present invention into the secretory pathway of the host cells, a secretory signal sequence (also known as a leader sequence, prepro sequence or pre sequence) may be provided in the recombinant vector. The secretory signal sequence is joined to the DNA sequence encoding the peptide in the correct reading frame. Secretory signal sequences are commonly positioned 5′ to the DNA sequence encoding the peptide. The secretory signal sequence may be that normally associated with the peptide or may be from a gene encoding another secreted protein.

For secretion from yeast cells, the secretory signal sequence may encode any signal peptide which ensures efficient direction of the expressed peptide into the secretory pathway of the cell. The signal peptide may be naturally occurring signal peptide, or a functional part thereof, or it may be a synthetic peptide. Suitable signal peptides have been found to be the α-factor signal peptide (cf. U.S. Pat. No. 4,870,008), the signal peptide of mouse salivary amylase (cf. O. Hagenbuchle et al., Nature 289 643-646 (1981)), a modified carboxypeptidase signal peptide (cf. L. A. Valls et al., Cell 48, 887-897 (1987)), the yeast BAR1 signal peptide (cf. WO 87/02670), or the yeast aspartic protease 3 (YAP3) signal peptide (cf. M. Egel-Mitani et al., Yeast 6, 127-137 (1990)).

For efficient secretion in yeast, a sequence encoding a leader peptide may also be inserted downstream of the signal sequence and uptream of the DNA sequence encoding the peptide. The function of the leader peptide is to allow the expressed peptide to be directed from the endoplasmic reticulum to the Golgi apparatus and further to a secretory vesicle for secretion into the culture medium (i.e. exportation of the peptide across the cell wall or at least through the cellular membrane into the periplasmic space of the yeast cell). The leader peptide may be the yeast α-factor leader (the use of which is described in e.g. U.S. Pat. No. 4,546,082, EP 16 201, EP 123 294, EP 123 544 and EP 163 529). Alternatively, the leader peptide may be a synthetic leader peptide, which is to say a leader peptide not found in nature. Synthetic leader peptides may, for instance, be constructed as described in WO 89/02463 or WO 92/11378.

For use in filamentous fungi, the signal peptide may conveniently be derived from a gene encoding an Aspergillus sp. amylase or glucoamylase, a gene encoding a Rhizomucor miehei lipase or protease or a Humicola lanuginosa lipase. The signal peptide may be derived from a gene encoding A. oryzae TAKA amylase, A. niger neutral α-amylase, A. niger acid-stable amylase, or A. niger glucoamylase.

The procedures used to ligate the DNA sequences coding for the present peptide, the promoter and optionally the terminator and/or secretory signal sequence, respectively, and to insert them into suitable vectors containing the information necessary for replication, are well known to persons skilled in the art (cf., for instance, Sambrook et al., op.cit.).

The host cell into which the DNA construct or the recombinant vector of the invention is introduced may be any cell which is capable of producing the present peptide and includes bacteria, yeast, fungi and higher eukaryotic cells.

Examples of bacterial host cells which, on cultivation, are capable of producing the peptide of the invention are grampositive bacteria such as strains of Bacillus, such as strains of B. subtilis, B. licheniformis, B. lentus, B. brevis, B. stearothermophilus, B. alkalophilus, B. amyloliquefaciens, B. coagulans, B. circulans, B. lautus, B. megatherium or B. thuringiensis, or strains of Streptomyces, such as S. lividans or S. murinus, or gramnegative bacteria such as Echerichia coli. The transformation of the bacteria may be effected by protoplast transformation or by using competent cells in a manner known per se (cf. Sambrook et al., supra). Other suitable hosts include S. mobaraense, S. lividans, and C. glutamicum (Appl. Microbiol. Biotechnol. 64, 447-454 (2004)).

When expressing the peptide in bacteria such as E. coli, the peptide may be retained in the cytoplasm, typically as insoluble granules (known as inclusion bodies), or may be directed to the periplasmic space by a bacterial secretion sequence. In the former case, the cells are lysed and the granules are recovered and denatured after which the peptide is refolded by diluting the denaturing agent. In the latter case, the peptide may be recovered from the periplasmic space by disrupting the cells, e.g. by sonication or osmotic shock, to release the contents of the periplasmic space and recovering the peptide.

Examples of suitable yeasts cells include cells of Saccharomyces spp. or Schizosaccharomyces spp., in particular strains of Saccharomyces cerevisiae or Saccharomyces kluyveri. Methods for transforming yeast cells with heterologous DNA and producing heterologous proteins therefrom are described, e.g. in U.S. Pat. No. 4,599,311, U.S. Pat. No. 4,931,373, U.S. Pat. Nos. 4,870,008, 5,037,743, and U.S. Pat. No. 4,845,075, all of which are hereby incorporated by reference. Transformed cells are selected by a phenotype determined by a selectable marker, commonly drug resistance or the ability to grow in the absence of a particular nutrient, e.g. leucine. An example of a vector for use in yeast is the POT1 vector disclosed in U.S. Pat. No. 4,931,373. The DNA sequence encoding the peptide of the invention may be preceded by a signal sequence and optionally a leader sequence, e.g. as described above. Further examples of suitable yeast cells are strains of Kluyveromyces, such as K. lactis, Hansenula, e.g. H. polymorpha, or Pichia, e.g. P. pastoris (cf. Gleeson et al., J. Gen. Microbiol. 132, 3459-3465 (1986); U.S. Pat. No. 4,882,279).

Examples of other fungal cells are cells of filamentous fungi, e.g. Aspergillus spp., Neurospora spp., Fusarium spp. or Trichoderma spp., in particular strains of A. oryzae, A. nidulans or A. niger. The use of Aspergillus spp. for the expression of proteins is described in, e.g., EP 272 277 and EP 230 023. The transformation of F. oxysporum may, for instance, be carried out as described by Malardier et al. Gene 78, 147-156 (1989).

When a filamentous fungus is used as the host cell, it may be transformed with the DNA construct of the invention, conveniently by integrating the DNA construct in the host chromosome to obtain a recombinant host cell. This will make it more likely that the DNA sequence will be stably maintained in the cell. Integration of the DNA constructs into the host chromosome may be performed according to conventional methods, e.g. by homologous or heterologous recombination.

The transformed or transfected host cell described above is then cultured in a suitable nutrient medium under conditions permitting the expression of the present peptide, after which the resulting peptide is recovered from the culture.

The medium used to culture the cells may be any conventional medium suitable for growing the host cells, such as minimal or complex media containing appropriate supplements. Suitable media are available from commercial suppliers or may be prepared according to published recipes (e.g. in catalogues of the American Type Culture Collection). The peptide produced by the cells may then be recovered from the culture medium by conventional procedures including separating the host cells from the medium by centrifugation or filtration, precipitating the proteinaceous components of the supernatant or filtrate by means of a salt, e.g. ammonium sulphate, purification by a variety of chromatographic procedures, e.g. ion exchange chromatography, gelfiltration chromatography, affinity chromatography, or the like, dependent on the type of peptide in question.

### Pharmaceutical Compositions

The present invention provides a pharmaceutical formulation comprising a peptide of the present invention which is present in a concentration from 10⁻¹⁵ mg/ml to 200 mg/ml, such as 10⁻¹⁰ mg/ml-5 mg/ml, and wherein said formulation has a pH from 2.0 to 10.0. Optionally, said formulation may comprise one or more further cancer agents as described above. The formulation may further comprise a buffer system, preservative(s), tonicity agent(s), chelating agent(s), stabilizers and surfactants. In one embodiment of the invention the pharmaceutical formulation is an aqueous formulation, i.e. formulation comprising water. Such formulation is typically a solution or a suspension. In one embodiment of the invention the pharmaceutical formulation is an aqueous solution. The term “aqueous formulation” is defined as a formulation comprising at least 50% w/w water. Likewise, the term “aqueous solution” is defined as a solution comprising at least 50% w/w water, and the term “aqueous suspension” is defined as a suspension comprising at least 50% w/w water.

In one embodiment the pharmaceutical formulation is a freeze-dried formulation, whereto the physician or the patient adds solvents and/or diluents prior to use.

In one embodiment the pharmaceutical formulation is a dried formulation (e.g. freeze-dried or spray-dried) ready for use without any prior dissolution.

In one embodiment the invention relates to a pharmaceutical formulation comprising an aqueous solution of a peptide of the present invention, and a buffer, wherein said peptide is present in a concentration from 0.1-100 mg/ml, and wherein said formulation has a pH from about 2.0 to about 10.0.

In one embodiment of the invention the pH of the formulation is selected from the list consisting of 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, and 10.0.

In one embodiment of the invention the buffer is selected from the group consisting of sodium acetate, sodium carbonate, citrate, glycylglycine, histidine, glycine, lysine, arginine, sodium dihydrogen phosphate, disodium hydrogen phosphate, sodium phosphate, and tris(hydroxymethyl)-aminomethan, bicine, tricine, malic acid, succinate, maleic acid, fumaric acid, tartaric acid, aspartic acid or mixtures thereof. Each one of these specific buffers constitutes an alternative embodiment of the invention.

In one embodiment of the invention the formulation further comprises a pharmaceutically acceptable preservative. In one embodiment of the invention the preservative is selected from the group consisting of phenol, o-cresol, m-cresol, p-cresol, methyl p-hydroxybenzoate, propyl p-hydroxybenzoate, 2-phenoxyethanol, butyl p-hydroxybenzoate, 2-phenylethanol, benzyl alcohol, chlorobutanol, and thiomerosal, bronopol, benzoic acid, imidurea, chlorohexidine, sodium dehydroacetate, chlorocresol, ethyl p-hydroxybenzoate, benzethonium chloride, chlorphenesine (3p-chlorphenoxypropane-1,2-diol) or mixtures thereof. In one embodiment of the invention the preservative is present in a concentration from 0.1 mg/ml to 20 mg/ml. In one embodiment of the invention the preservative is present in a concentration from 0.1 mg/ml to 5 mg/ml. In one embodiment of the invention the preservative is present in a concentration from 5 mg/ml to 10 mg/ml. In one embodiment of the invention the preservative is present in a concentration from 10 mg/ml to 20 mg/ml. Each one of these specific preservatives constitutes an alternative embodiment of the invention. The use of a preservative in pharmaceutical compositions is well-known to the skilled person. For convenience reference is made to Remington: The Science and Practice of Pharmacy, 20^{th} edition, 2000.

In one embodiment of the invention the formulation further comprises an isotonic agent. In one embodiment of the invention the isotonic agent is selected from the group consisting of a salt (e.g. sodium chloride), a sugar or sugar alcohol, an amino acid (e.g. L-glycine, L-histidine, arginine, lysine, isoleucine, aspartic acid, tryptophan, threonine), an alditol (e.g. glycerol (glycerine), 1,2-propanediol (propyleneglycol), 1,3-propanediol, 1,3-butanediol) polyethyleneglycol (e.g. PEG400), or mixtures thereof. Any sugar such as mono-, di-, or polysaccharides, or water-soluble glucans, including for example fructose, glucose, mannose, sorbose, xylose, maltose, lactose, sucrose, trehalose, dextran, pullulan, dextrin, cyclodextrin, soluble starch, hydroxyethyl starch and carboxymethylcellulose-Na may be used. In one embodiment the sugar additive is sucrose. Sugar alcohol is defined as a C4-C8 hydrocarbon having at least one —OH group and includes, for example, mannitol, sorbitol, inositol, galactitol, dulcitol, xylitol, and arabitol. In one embodiment the sugar alcohol additive is mannitol. The sugars or sugar alcohols mentioned above may be used individually or in combination. There is no fixed limit to the amount used, as long as the sugar or sugar alcohol is soluble in the liquid preparation and does not adversely effect the stabilizing effects achieved using the methods of the invention. In one embodiment, the sugar or sugar alcohol concentration is between about 1 mg/ml and about 150 mg/ml. In one embodiment of the invention the isotonic agent is present in a concentration from 1 mg/ml to 50 mg/ml. In one embodiment of the invention the isotonic agent is present in a concentration from 1 mg/ml to 7 mg/ml. In one embodiment of the invention the isotonic agent is present in a concentration from 8 mg/ml to 24 mg/ml. In one embodiment of the invention the isotonic agent is present in a concentration from 25 mg/ml to 50 mg/ml. Each one of these specific isotonic agents constitutes an alternative embodiment of the invention. The use of an isotonic agent in pharmaceutical compositions is well-known to the skilled person. For convenience reference is made to Remington: The Science and Practice of Pharmacy, 20^{th} edition, 2000.

In one embodiment of the invention the formulation further comprises a chelating agent. In one embodiment of the invention the chelating agent is selected from salts of ethylenediaminetetraacetic acid (EDTA), citric acid, and aspartic acid, and mixtures thereof. In one embodiment of the invention the chelating agent is present in a concentration from 0.1 mg/ml to 5 mg/ml. In one embodiment of the invention the chelating agent is present in a concentration from 0.1 mg/ml to 2 mg/ml. In one embodiment of the invention the chelating agent is present in a concentration from 2 mg/ml to 5 mg/ml. Each one of these specific chelating agents constitutes an alternative embodiment of the invention. The use of a chelating agent in pharmaceutical compositions is well-known to the skilled person. For convenience reference is made to Remington: The Science and Practice of Pharmacy, 20^{th} edition, 2000.

In one embodiment of the invention the formulation further comprises a stabilizer. The use of a stabilizer in pharmaceutical compositions is well-known to the skilled person. For convenience reference is made to Remington: The Science and Practice of Pharmacy, 20^{th} edition, 2000.

More particularly, compositions of the invention are stabilized liquid pharmaceutical compositions whose therapeutically active components include a polypeptide that possibly exhibits aggregate formation during storage in liquid pharmaceutical formulations. By “aggregate formation” is intended a physical interaction between the polypeptide molecules that results in formation of oligomers, which may remain soluble, or large visible aggregates that precipitate from the solution. By “during storage” is intended a liquid pharmaceutical composition or formulation once prepared, is not immediately administered to a subject. Rather, following preparation, it is packaged for storage, either in a liquid form, in a frozen state, or in a dried form for later reconstitution into a liquid form or other form suitable for administration to a subject. By “dried form” is intended the liquid pharmaceutical composition or formulation is dried either by freeze drying (i.e., lyophilization; see, for example, Williams and Polli (1984) J. Parenteral Sci. Technol. 38:48-59), spray drying (see Masters (1991) in Spray-Drying Handbook (5th ed; Longman Scientific and Technical, Essez, U.K.), pp. 491-676; Broadhead et al. (1992) Drug Devel. Ind. Pharm. 18:1169-1206; and Mumenthaler et al. (1994) Pharm. Res. 11:12-20), or air drying (Carpenter and Crowe (1988) Cryobiology 25:459-470; and Roser (1991) Biopharm. 4:47-53). Aggregate formation by a polypeptide during storage of a liquid pharmaceutical composition can adversely affect biological activity of that polypeptide, resulting in loss of therapeutic efficacy of the pharmaceutical composition. Furthermore, aggregate formation may cause other problems such as blockage of tubing, membranes, or pumps when the polypeptide-containing pharmaceutical composition is administered using an infusion system.

The pharmaceutical compositions of the invention may further comprise an amount of an amino acid base sufficient to decrease aggregate formation by the polypeptide during storage of the composition. By “amino acid base” is intended an amino acid or a combination of amino acids, where any given amino acid is present either in its free base form or in its salt form. Where a combination of amino acids is used, all of the amino acids may be present in their free base forms, all may be present in their salt forms, or some may be present in their free base forms while others are present in their salt forms. In one embodiment, amino acids to use in preparing the compositions of the invention are those carrying a charged side chain, such as arginine, lysine, aspartic acid, and glutamic acid. Any stereoisomer (i.e., L, D, or mixtures thereof) of a particular amino acid (e.g. glycine, methionine, histidine, imidazole, arginine, lysine, isoleucine, aspartic acid, tryptophan, threonine and mixtures thereof) or combinations of these stereoisomers, may be present in the pharmaceutical compositions of the invention so long as the particular amino acid is present either in its free base form or its salt form. In one embodiment the L-stereoisomer is used. Compositions of the invention may also be formulated with analogues of these amino acids. By “amino acid analogue” is intended a derivative of the naturally occurring amino acid that brings about the desired effect of decreasing aggregate formation by the polypeptide during storage of the liquid pharmaceutical compositions of the invention. Suitable arginine analogues include, for example, aminoguanidine, ornithine and N-monoethyl L-arginine, suitable methionine analogues include ethionine and buthionine and suitable cysteine analogues include S-methyl-L cysteine. As with the other amino acids, the amino acid analogues are incorporated into the compositions in either their free base form or their salt form. In one embodiment of the invention the amino acids or amino acid analogues are used in a concentration, which is sufficient to prevent or delay aggregation of the protein.

In one embodiment of the invention methionine (or other sulphuric amino acids or amino acid analogous) may be added to inhibit oxidation of methionine residues to methionine sulfoxide when the polypeptide acting as the therapeutic agent is a polypeptide comprising at least one methionine residue susceptible to such oxidation. By “inhibit” is intended minimal accumulation of methionine oxidized species over time. Inhibiting methionine oxidation results in greater retention of the polypeptide in its proper molecular form. Any stereoisomer of methionine (L, D, or mixtures thereof) or combinations thereof can be used. The amount to be added should be an amount sufficient to inhibit oxidation of the methionine residues such that the amount of methionine sulfoxide is acceptable to regulatory agencies. Typically, this means that the composition contains no more than about 10% to about 30% methionine sulfoxide. Generally, this can be achieved by adding methionine such that the ratio of methionine added to methionine residues ranges from about 1:1 to about 1000:1, such as 10:1 to about 100:1.

In one embodiment of the invention the formulation further comprises a stabilizer selected from the group of high molecular weight polymers or low molecular compounds. In one embodiment of the invention the stabilizer is selected from polyethylene glycol (e.g. PEG 3350), polyvinyl alcohol (PVA), polyvinylpyrrolidone, carboxy-/hydroxycellulose or derivates thereof (e.g. HPC, HPC-SL, HPC-L and HPMC), cyclodextrins, sulphur-containing substances as monothioglycerol, thioglycolic acid and 2-methylthioethanol, and different salts (e.g. sodium chloride). Each one of these specific stabilizers constitutes an alternative embodiment of the invention.

The pharmaceutical compositions may also comprise additional stabilizing agents, which further enhance stability of a therapeutically active polypeptide therein. Stabilizing agents of particular interest to the present invention include, but are not limited to, methionine and EDTA, which protect the polypeptide against methionine oxidation, and a nonionic surfactant, which protects the polypeptide against aggregation associated with freeze-thawing or mechanical shearing.

In one embodiment of the invention the formulation further comprises a surfactant. In one embodiment of the invention the surfactant is selected from a detergent, ethoxylated castor oil, polyglycolyzed glycerides, acetylated monoglycerides, sorbitan fatty acid esters, polyoxypropylene-polyoxyethylene block polymers (eg. poloxamers such as Pluronic® F68, poloxamer 188 and 407, Triton X-100), polyoxyethylene sorbitan fatty acid esters, polyoxyethylene and polyethylene derivatives such as alkylated and alkoxylated derivatives (tweens, e.g. Tween-20, Tween-40, Tween-80 and Brij-35), monoglycerides or ethoxylated derivatives thereof, diglycerides or polyoxyethylene derivatives thereof, alcohols, glycerol, lectins and phospholipids (eg. phosphatidyl serine, phosphatidyl choline, phosphatidyl ethanolamine, phosphatidyl inositol, diphosphatidyl glycerol and sphingomyelin), derivates of phospholipids (eg. dipalmitoyl phosphatidic acid) and lysophospholipids (eg. palmitoyl lysophosphatidyl-L-serine and 1-acyl-sn-glycero-3-phosphate esters of ethanolamine, choline, serine or threonine) and alkyl, alkoxyl (alkyl ester), alkoxy (alkyl ether)-derivatives of lysophosphatidyl and phosphatidylcholines, e.g. lauroyl and myristoyl derivatives of lysophosphatidylcholine, dipalmitoylphosphatidylcholine, and modifications of the polar head group, that is cholines, ethanolamines, phosphatidic acid, serines, threonines, glycerol, inositol, and the positively charged DODAC, DOTMA, DCP, BISHOP, lysophosphatidylserine and lysophosphatidylthreonine, and glycerophospholipids (eg. cephalins), glyceroglycolipids (eg. galactopyransoide), sphingoglycolipids (eg. ceramides, gangliosides), dodecylphosphocholine, hen egg lysolecithin, fusidic acid derivatives-(e.g. sodium tauro-dihydrofusidate etc.), long-chain fatty acids and salts thereof C6-C12 (eg. oleic acid and caprylic acid), acylcarnitines and derivatives, N^{α}-acylated derivatives of lysine, arginine or histidine, or side-chain acylated derivatives of lysine or arginine, N^{α}-acylated derivatives of dipeptides comprising any combination of lysine, arginine or histidine and a neutral or acidic amino acid, N^{α}-acylated derivative of a tripeptide comprising any combination of a neutral amino acid and two charged amino acids, DSS (docusate sodium, CAS registry no [577-11-7]), docusate calcium, CAS registry no [128-49-4]), docusate potassium, CAS registry no [7491-09-0]), SDS (sodium dodecyl sulphate or sodium lauryl sulphate), sodium caprylate, cholic acid or derivatives thereof, bile acids and salts thereof and glycine or taurine conjugates, ursodeoxycholic acid, sodium cholate, sodium deoxycholate, sodium taurocholate, sodium glycocholate, N-Hexadecyl-N,N-dimethyl-3-ammonio-1-propanesulfonate, anionic (alkyl-aryl-sulphonates) monovalent surfactants, zwitterionic surfactants (e.g. N-alkyl-N,N-dimethylammonio-1-propanesulfonates, 3-cholamido-1-propyldimethylammonio-1-propanesulfonate, cationic surfactants (quaternary ammonium bases) (e.g. cetyl-trimethylammonium bromide, cetylpyridinium chloride), non-ionic surfactants (eg. Dodecyl β-D-glucopyranoside), poloxamines (eg. Tetronic's), which are tetrafunctional block copolymers derived from sequential addition of propylene oxide and ethylene oxide to ethylenediamine, or the surfactant may be selected from the group of imidazoline derivatives, or mixtures thereof. Each one of these specific surfactants constitutes an alternative embodiment of the invention.

The use of a surfactant in pharmaceutical compositions is well-known to the skilled person. For convenience reference is made to Remington: The Science and Practice of Pharmacy, 20^{th} edition, 2000.

It is possible that other ingredients may be present in the peptide pharmaceutical formulation of the present invention. Such additional ingredients may include wetting agents, emulsifiers, antioxidants, bulking agents, tonicity modifiers, chelating agents, metal ions, oleaginous vehicles, proteins (e.g., human serum albumin, gelatine or proteins) and a zwitterion (e.g., an amino acid such as betaine, taurine, arginine, glycine, lysine and histidine). Such additional ingredients, of course, should not adversely affect the overall stability of the pharmaceutical formulation of the present invention.

Pharmaceutical compositions containing a peptide of the present invention may be administered to a patient in need of such treatment at several sites, for example, at topical sites, for example, skin and mucosal sites, at sites which bypass absorption, for example, administration in an artery, in a vein, in the heart, and at sites which involve absorption, for example, administration in the skin, under the skin, in a muscle or in the abdomen.

Administration of pharmaceutical compositions according to the invention may be through several routes of administration, for example, lingual, sublingual, buccal, in the mouth, oral, in the stomach and intestine, nasal, pulmonary, for example, through the bronchioles and alveoli or a combination thereof, epidermal, dermal, transdermal, vaginal, rectal, ocular, for examples through the conjunctiva, uretal, and parenteral to patients in need of such a treatment.

Compositions of the current invention may be administered in several dosage forms, for example, as solutions, suspensions, emulsions, microemulsions, multiple emulsion, foams, salves, pastes, plasters, ointments, tablets, coated tablets, rinses, capsules, for example, hard gelatine capsules and soft gelatine capsules, suppositories, rectal capsules, drops, gels, sprays, powder, aerosols, inhalants, eye drops, ophthalmic ointments, ophthalmic rinses, vaginal pessaries, vaginal rings, vaginal ointments, injection solution, in situ transforming solutions, for example in situ gelling, in situ setting, in situ precipitating, in situ crystallization, infusion solution, and implants.

Compositions of the invention may further be compounded in, or attached to, for example through covalent, hydrophobic and electrostatic interactions, a drug carrier, drug delivery system and advanced drug delivery system in order to further enhance stability of the peptide of the present invention, increase bioavailability, increase solubility, decrease adverse effects, achieve chronotherapy well known to those skilled in the art, and increase patient compliance or any combination thereof. Examples of carriers, drug delivery systems and advanced drug delivery systems include, but are not limited to, polymers, for example cellulose and derivatives, polysaccharides, for example dextran and derivatives, starch and derivatives, poly(vinyl alcohol), acrylate and methacrylate polymers, polylactic and polyglycolic acid and block co-polymers thereof, polyethylene glycols, carrier proteins, for example albumin, gels, for example, thermogelling systems, for example block co-polymeric systems well known to those skilled in the art, micelles, liposomes, microspheres, nanoparticulates, liquid crystals and dispersions thereof, L2 phase and dispersions there of, well known to those skilled in the art of phase behaviour in lipid-water systems, polymeric micelles, multiple emulsions, self-emulsifying, self-microemulsifying, cyclodextrins and derivatives thereof, and dendrimers.

Compositions of the current invention are useful in the formulation of solids, semisolids, powder and solutions for pulmonary administration of a peptide of the present invention, using, for example a metered dose inhaler, dry powder inhaler and a nebulizer, all being devices well known to those skilled in the art.

Compositions of the current invention are specifically useful in the formulation of controlled, sustained, protracting, retarded, and slow release drug delivery systems. More specifically, but not limited to, compositions are useful in formulation of parenteral controlled release and sustained release systems (both systems leading to a many-fold reduction in number of administrations), well known to those skilled in the art. Even more preferably, are controlled release and sustained release systems administered subcutaneous. Without limiting the scope of the invention, examples of useful controlled release system and compositions are hydrogels, oleaginous gels, liquid crystals, polymeric micelles, microspheres, nanoparticles,

Methods to produce controlled release systems useful for compositions of the current invention include, but are not limited to, crystallization, condensation, co-crystallization, precipitation, co-precipitation, emulsification, dispersion, high pressure homogenisation, encapsulation, spray drying, microencapsulating, coacervation, phase separation, solvent evaporation to produce microspheres, extrusion and supercritical fluid processes. General reference is made to Handbook of Pharmaceutical Controlled Release (Wise, D. L., ed. Marcel Dekker, New York, 2000) and Drug and the Pharmaceutical Sciences vol. 99: Protein Formulation and Delivery (MacNally, E. J., ed. Marcel Dekker, New York, 2000).

Parenteral administration may be performed by subcutaneous, intramuscular, intraperitoneal or intravenous injection by means of a syringe, optionally a pen-like syringe. Alternatively, parenteral administration can be performed by means of an infusion pump. A further option is a composition which may be a solution or suspension for the administration of the peptide of the present invention in the form of a nasal or pulmonal spray. As a still further option, the pharmaceutical compositions containing the peptide of the present invention can also be adapted to transdermal administration, e.g. by needle-free injection or from a patch, optionally an iontophoretic patch, or transmucosal, e.g. buccal, administration.

The following is a non-limiting list of embodiments of the present invention.

### Embodiment 1

An isolated peptide, which peptide is prolactin or a prolactin analogue, and which binds to the prolactin receptor, said peptide having an amino acid mutation, which mutation results in altered secondary structure at a position corresponding to positions 55, 56 or 57 of SEQ ID No. 1, wherein said method comprises mutating one or more amino acids in the segment corresponding to positions 47-57 of SEQ ID No. 1.

### Embodiment 2

An isolated peptide, which peptide is a prolactin analogue, which binds to the prolactin receptor, said peptide having an amino acid mutation in the position corresponding to position 55, 56 or 57 of SEQ ID No. 1.

### Embodiment 3

An isolated peptide, which peptide is a prolactin analogue which binds to the prolactin receptor, said peptide comprising the amino acid sequence of SEQ ID No. 1 having an amino acid mutation in the position corresponding to position 55, 56 or 57 of SEQ ID No. 1.

### Embodiment 4

An isolated peptide according to any of embodiments 1 to 3 having an amino acid mutation in the position corresponding to position 55 of SEQ ID No. 1.

### Embodiment 5

An isolated peptide according to any of embodiments 1 to 4, wherein the amino acid residue in the position corresponding to position 55 of SEQ ID No. 1 has been substituted with an amino acid residue that suppresses deamidation of the amino acid residue in position 56 of SEQ ID No. 1.

### Embodiment 6

An isolated peptide according to embodiment 5, wherein the amino acid residue in the position corresponding to position 55 of SEQ ID No. 1 has been substituted with an amino acid residue other than isoleucine.

### Embodiment 7

An isolated peptide according to embodiment 6, wherein the amino acid residue in the position corresponding to position 55 of SEQ ID No. 1 has been substituted with an amino acid residue other than isoleucine or glycine.

### Embodiment 8

An isolated peptide according to embodiment 7, wherein the amino acid residue in the position corresponding to position 55 of SEQ ID No. 1 has been substituted with valine, leucine, tryptophan, tyrosine, phenylalanine, proline or threonine.

### Embodiment 9

An isolated peptide according to any of embodiments 1 to 3 having an amino acid mutation in the position corresponding to position 56 of SEQ ID No. 1.

### Embodiment 10

An isolated peptide according to embodiment 9, wherein the amino acid residue in the position corresponding to position 56 of SEQ ID No. 1 has been substituted with an amino acid residue that is resistant to deamidation.

### Embodiment 11

An isolated peptide according to embodiment 10, wherein the amino acid residue in the position corresponding to position 56 of SEQ ID No. 1 has been substituted with glycine.

### Embodiment 12

An isolated peptide according to embodiment 10, wherein the amino acid residue in the position corresponding to position 56 of SEQ ID No. 1 has been substituted with glutamine.

### Embodiment 13

An isolated peptide according to any of embodiments 1 to 3 having an amino acid mutation in the position corresponding to position 57 of SEQ ID No. 1.

### Embodiment 14

An isolated peptide according to embodiment 13, wherein the amino acid residue in the position corresponding to position 57 of SEQ ID No. 1 has been substituted with an amino acid residue that suppresses deamidation of the amino acid residue in position 56 of SEQ ID No. 1.

### Embodiment 15

An isolated peptide according to embodiment 14, wherein the amino acid residue in the position corresponding to position 57 of SEQ ID No. 1 has been substituted with any of the natural amino acids encoded by the genetic code other than serine or glycine.

### Embodiment 16

An isolated peptide according to embodiment 15, wherein the amino acid residue in the position corresponding to position 57 of SEQ ID No. 1 has been substituted with valine, leucine, isoleucine, tryptophan, tyrosine, phenylalanine, proline or threonine.

### Embodiment 17

An isolated peptide according to any of embodiments 1 to 16, which is an antagonist of the prolactin receptor.

### Embodiment 18

An isolated peptide according to embodiment 17, wherein said antagonism is achieved by introducing one or more mutations to prevent or reduce interaction of BS2 with the prolactin receptor.

### Embodiment 19

An isolated peptide according to embodiment 17 or embodiment 18, wherein at least one or more of said antagonistic mutations are selected from mutations in the amino acid residues corresponding to Gly-129 and Ser-179.

### Embodiment 20

An isolated peptide according to embodiment 19, wherein at least one or more of said antagonistic mutations are selected from a mutation of the amino acid residue in the position corresponding to Gly-129 to an Arg and a mutation of the amino acid residue in the position corresponding to S179-D to an Asp.

### Embodiment 21

An isolated peptide according to embodiment 20, wherein at least one or more of said antagonistic mutations are selected from a mutation corresponding to G129R.

### Embodiment 22

An isolated peptide according to any of embodiments 17 to 21, wherein the amino acid residues corresponding to positions 1 to 9 in SEQ ID No. 1 have been deleted.

### Embodiment 23

An isolated peptide according to any of embodiments 17 to 22, wherein the amino acid residues corresponding to positions 1 to 14 in SEQ ID No. 1 have been deleted.

### Embodiment 24

An isolated peptide according to any of embodiments 1 to 16, which is an agonist of the prolactin receptor.

### Embodiment 25

An isolated peptide according to embodiment 24, wherein said peptide binds BS2.

### Embodiment 26

An isolated nucleic acid encoding a peptide according to any of embodiments 1 to 25.

### Embodiment 27

A vector comprising a nucleic acid construct according to embodiment 26.

### Embodiment 28

A host cell comprising a nucleic acid construct of embodiment 26, or a vector of embodiment 27.

### Embodiment 29

An antibody that specifically binds a peptide according to any of embodiments 1 to 25.

### Embodiment 30

An antibody according to embodiment 29, which antibody does not bind to a peptide comprising the amino acid sequence of SEQ ID No. 1.

### Embodiment 31

A method for stabilising prolactin or a prolactin analogue comprising mutating an amino acid in a position resulting in induction of secondary structure at a position corresponding to positions 55, 56 or 57 of SEQ ID No. 1, wherein said method comprises mutating one or more amino acids in the segment corresponding to positions 47-57 of SEQ ID No. 1.

### Embodiment 32

A method for stabilising prolactin or a prolactin analogue comprising mutating an amino acid in a position resulting in induction of secondary structure at a position corresponding to positions 55, 56 or 57 of SEQ ID No. 1.

### Embodiment 33

A method according to embodiment 32 which comprises mutating an amino acid corresponding to positions 55, 56 or 57 of SEQ ID No. 1.

### Embodiment 34

A method according to embodiment 33 having an amino acid mutation in the position corresponding to position 56 of SEQ ID No. 1.

### Embodiment 35

A method according to embodiment 34, wherein the amino acid residue in the position corresponding to position 56 of SEQ ID No. 1 has been substituted with an amino acid residue that is resistant to deamidation.

### Embodiment 36

A method according to embodiment 35, wherein the amino acid residue in the position corresponding to position 56 of SEQ ID No. 1 has been substituted with glycine.

### Embodiment 37

A method according to embodiment 32 which comprises mutating an amino acid corresponding to positions 55 or 57 of SEQ ID No. 1.

### Embodiment 38

A method according to embodiment 37 having an amino acid mutation in the position corresponding to position 55 of SEQ ID No. 1.

### Embodiment 39

A method according to embodiment 37 or 38, wherein the amino acid residue in the position corresponding to position 55 of SEQ ID No. 1 has been substituted with an amino acid residue that suppresses deamidation of the amino acid residue in position 56 of SEQ ID No. 1.

### Embodiment 40

A method according to embodiment 39, wherein the amino acid residue in the position corresponding to position 55 of SEQ ID No. 1 has been substituted with an amino acid residue other than isoleucine.

### Embodiment 41

A method according to embodiment 40, wherein the amino acid residue in the position corresponding to position 55 of SEQ ID No. 1 has been substituted with valine, leucine, tryptophan, tyrosine, phenylalanine, proline or threonine.

### Embodiment 42

A method according to any of embodiments 32 to 41 having an amino acid mutation in the position corresponding to position 57 of SEQ ID No. 1.

### Embodiment 43

A method according to embodiment 42, wherein the amino acid residue in the position corresponding to position 57 of SEQ ID No. 1 has been substituted with an amino acid residue that suppresses deamidation of the amino acid residue in position 56 of SEQ ID No. 1.

### Embodiment 44

A method according to embodiment 43, wherein the amino acid residue in the position corresponding to position 57 of SEQ ID No. 1 has been substituted with any of the natural amino acids encoded by the genetic code other than serine or glycine.

### Embodiment 45

A method according to embodiment 44, wherein the amino acid residue in the position corresponding to position 57 of SEQ ID No. 1 has been substituted with valine, leucine, isoleucine, tryptophan, tyrosine, phenylalanine, proline or threonine.

### Embodiment 46

A peptide according to any of embodiments 1 to 25 for use in therapy.

### Embodiment 47

A peptide according to embodiment 46 for use in treating or preventing a proliferative disorder.

### Embodiment 48

A peptide according to embodiment 47, wherein said proliferative disorder is a cancer.

### Embodiment 49

A peptide according to embodiment 48, wherein said cancer is selected from an estrogen dependent cancer, breast cancer, prostate cancer, lung cancer, colorectal cancer, head and neck cancer, ovarian cancer, cervical cancer, bladder cancer, pancreatic cancer, gastrointestinal cancer, leukaemia, skin cancer, and lymphoma.

### Embodiment 50

A peptide according to embodiment 49, wherein said cancer is breast, prostate, colorectal, head, neck or lung cancer.

### Embodiment 51

A peptide according to embodiment 50, wherein said cancer is breast cancer.

### Embodiment 52

A peptide according to any of embodiments 46 to 51 for use alone or in combination with anti-estrogen therapies.

### Embodiment 53

A peptide according to any of embodiments 46 to 51 for use alone or in combination with inhibitors of growth factor receptors signalling.

### Embodiment 54

A peptide according to any of embodiments 46 to 51 for use alone or in combination with anti-angiogenesis therapies.

### Embodiment 55

A peptide according to any of embodiments 46 to 51 for use alone or in combination with anti-lymphogenic therapies.

### Embodiment 56

A peptide according to any of embodiments 46 to 51 for use alone or in combination with immunomodulating therapies.

### Embodiment 57

A peptide according to any of embodiments 46 to 51 for use alone or in combination with chemotherapeutic agents.

### Embodiment 58

A pharmaceutical formulation comprising a peptide according to any of embodiments 1 to 25.

### Embodiment 59

A pharmaceutical formulation according to embodiment 58 for use in the treatment or prevention of a proliferative disorder.

### Embodiment 60

A pharmaceutical formulation according to embodiment 59, wherein said proliferative disorder is a cancer.

### Embodiment 61

A pharmaceutical formulation according to embodiment 60, wherein said cancer is selected from an estrogen dependent cancer, breast cancer, prostate cancer, lung cancer, colorectal cancer, head and neck cancer, ovarian cancer, cervical cancer, bladder cancer, pancreatic cancer, gastrointestinal cancer, leukaemia, skin cancer, and lymphoma.

### Embodiment 62

A pharmaceutical formulation according to embodiment 61, wherein said cancer is breast cancer.

### Embodiment 63

Use of a peptide according to any of embodiments 1 to 25 for therapy.

### Embodiment 64

Use of a peptide according to any of embodiments 1 to 25 in the treatment or prevention of a proliferative disorder.

### Embodiment 65

Use of a peptide according to any of embodiments 1 to 25 for the preparation of a pharmaceutical composition for the treatment or prevention of a proliferative disorder.

### Embodiment 66

Use according to embodiment 64 or embodiment 65, wherein said proliferative disorder is a cancer.

### Embodiment 67

Use according to embodiment 66, wherein said cancer is selected from an estrogen dependent cancer, breast cancer, prostate cancer, lung cancer, colorectal cancer, head and neck cancer, ovarian cancer, cervical cancer, bladder cancer, pancreatic cancer, gastrointestinal cancer, leukaemia, skin cancer, and lymphoma.

### Embodiment 68

A use according to embodiment 67, wherein said cancer is breast, prostate, colorectal, head, neck or lung cancer.

### Embodiment 69

Use according to embodiment 68, wherein said cancer is breast cancer.

### Embodiment 70

A method of treatment or prevention of a proliferative disorder, which comprises administration of an effective amount of a peptide according to any of embodiments 1 to 18 or a pharmaceutical formulation according to any of embodiments 58 to 62 to a patient in need thereof.

### Embodiment 71

A method according to embodiment 70, wherein said proliferative disorder is a cancer.

### Embodiment 72

A method according to embodiment 71, wherein said cancer is selected from an estrogen dependent cancer, breast cancer, prostate cancer, lung cancer, colorectal cancer, head and neck cancer, ovarian cancer, cervical cancer, bladder cancer, pancreatic cancer, gastrointestinal cancer, leukaemia, skin cancer, and lymphoma.

### Embodiment 73

A method according to embodiment 72, wherein said cancer is breast, prostate, colorectal, head, neck or lung cancer.

### Embodiment 74

A method according to embodiment 73, wherein said cancer is breast cancer.

### Embodiment 75

Use of a peptide according to any of embodiments 1 to 25 or a nucleic acid according to embodiment 26 for generating prolactin antagonists for the treatment of proliferative disorders.

### Embodiment 76

A use according to embodiment 75, wherein said proliferative disorder is a cancer.

### Embodiment 77

A use according to embodiment 76, wherein said cancer is selected from an estrogen dependent cancer, breast cancer, prostate cancer, lung cancer, colorectal cancer, head and neck cancer, ovarian cancer, cervical cancer, bladder cancer, pancreatic cancer, gastrointestinal cancer, leukaemia, skin cancer, and lymphoma.

### Embodiment 78

A use according to embodiment 77, wherein said cancer is breast, prostate, colorectal, head, neck or lung cancer.

### Embodiment 79

A use according to embodiment 78, wherein said cancer is breast cancer.

All references, including publications, patent applications, and patents, cited herein are hereby incorporated by reference in their entirety and to the same extent as if each reference were individually and specifically indicated to be incorporated by reference and were set forth in its entirety herein (to the maximum extent permitted by law), regardless of any separately provided incorporation of particular documents made elsewhere herein.

The use of the terms “a” and “an” and “the” and similar referents in the context of describing the invention are to be construed to cover both the singular and the plural, unless otherwise indicated herein or clearly contradicted by context. For example, the phrase “the compound” is to be understood as referring to various “compounds” of the invention or particular described aspect, unless otherwise indicated.

Unless otherwise indicated, all exact values provided herein are representative of corresponding approximate values (e.g., all exact exemplary values provided with respect to a particular factor or measurement can be considered to also provide a corresponding approximate measurement, modified by “about,” where appropriate).

The description herein of any aspect or aspect of the invention using terms such as “comprising”, “having,” “including,” or “containing” with reference to an element or elements is intended to provide support for a similar aspect or aspect of the invention that “consists of”, “consists essentially of”, or “substantially comprises” that particular element or elements, unless otherwise stated or clearly contradicted by context (e.g., a composition described herein as comprising a particular element should be understood as also describing a composition consisting of that element, unless otherwise stated or clearly contradicted by context).

### Example 1

### Identification of BS1 in PRL

### Summary:

Wild-type prolactin (wtPRL) was incubated in phosphate-buffer (pH 8) at 40° C. in order to induce deamidation. After incubation for 4 days individual deamidation products were isolated using ion exchange chromatography. One main and three minor deamidation products were isolated and characterized by peptide-mapping. Deamidation in the major degradation product was demonstrated to have taken place at Asn-56. It has not been possible to map the exact site(s) of deamidation in the three minor degradation products. These sites are most likely located in the very C-terminal part of the protein and furthermore one of the minor degradation product is resulting from di-deamidation.

### Methods:

Preparative ion exchange chromatography. Preparative cation exchange chromatography was performed using a MonoQ 5/50 GL column and a flow of 1 ml/min. The sample was eluted using a gradient of 5-15% buffer B over 30 minutes, where buffer A was 20 mM tris, pH 8.0 and buffer B 20 mM tris, pH 8.0, 1 M NaCl.

Mass spectrometry. A Bruker Microflex MALDI-MS instrument (Bremen, Germany) was used for peptide-mapping. Preferred matrix for peptide-mapping was alfa-cyano cinnamic acid. Sample application and analysis followed standard procedures.

Peptide mapping. Enzymatic digestion was performed in the incubation buffer used for inducing deamidation at an E:S ratio of 1:100 with overnight incubation at 37° C. The digest mixture was applied directly to the MALDI-MS target plate and analysed as outlined above. Asp-N and Trypsin was obtained from Roche Diagnostics, Mannheim, Germany.

### Results:

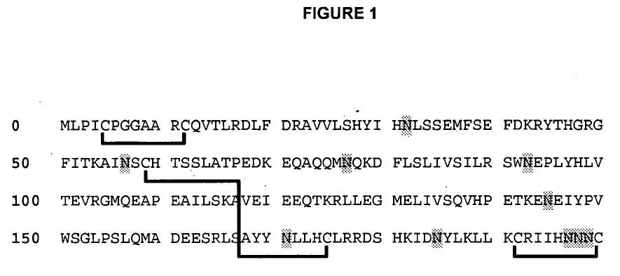

Sequence of wtPRL with possible deamidation-sites (Asn-residues) highlighted is shown in FIG. 1. The theoretical MW is: 23023.4 amu

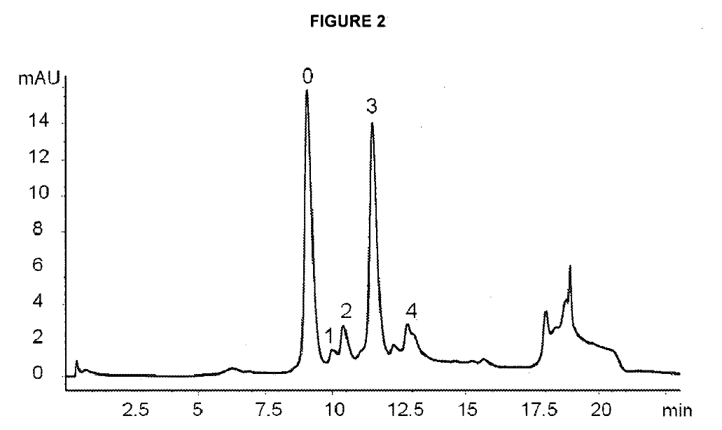

A 5 mg/ml solution of wPRL was incubated in a phosphate-buffer, pH 8 at elevated temperature (40° C.) in order to induce deamidation. FIG. 2 shows the anion exchange chromatogram following incubation for 4 days.

The individual peaks were collected and subjected to direct LC-MS analysis. The native wtPRL is the only significant peak present at the start of the deamidation experiment. All experimentally obtained masses were identical to the theoretically expected value for un-modified wtPRL (23023.4 amu). The accuracy of this molecular weight determination is approximately 0.02% corresponding to ±4 amu at 20000 amu. Thus, it is not possible from the measured molecular weights to distinguish deamidated from non-deamiated species (deamidation at a single Asn-residue will result in a mass increase of 1 amu). The results do, however, confirm that deamidation is the most probable cause of the observed degradation, as other modifications would potentially result in higher mass differences.

The individual peaks were subsequently subjected to peptide-mapping using trypsin and Asp-N followed by direct MALDI-MS analysis. The tryptic peptide-maps based on direct MALDI-MS cover seven out of the total ten Asn-residues. The residues that are not covered are all located in the C-terminal part of the protein (Asn-184, Asn-196, Asn-197 and Asn-198). One of the peptides detected in the MALDI-MS analysis displays a mass shift of +1 Da. This peptide (m/z 2759.7 in the spectrum of peak 0) corresponds to the tryptic peptide AA 54-78, which contains two Asn-residues (Asn-56 and Asn-76). When these data were combined, the site of deamidation in peak 3 was determined to Asn-56.

A similar Asp-N peptide map gives information of the C-terminal four Asn-residues not covered by the tryptic peptide map. From these spectra it can be concluded that peak 1 and 4 display a one Dalton mass shift for the AA 182-199 peptide.

In the preparative chromatography peak 2 is not well separated from peak 3 and the results for peak 2 are therefore not clear. It appears that a one Dalton mass shift may also be observed in peak 2 for the AA 182-199 peptide.

### Conclusion:

In conclusion the following has been demonstrated regarding the degradation products isolated by ion exchange chromatography:
1. Major degradation product (peak 3): deamidation at Asn-56
2. Minor degradation product (peak 1): deamidation in AA 182-199 (Asn-184, Asn-196, Asn-197 or Asn-198)
3. Minor degradation product (peak 4): deamidation at Asn-56 and in AA 182-199 (Asn-184, Asn-196, Asn-197 and Asn-198)
4. Minor degradation product (peak 2): possible deamidation in AA 182-199, but contamination with major degradation product (peak 3) makes this result uncertain.

### Example 2

### PRL receptor binding affinity of N56D,G129R-PRL

### Expresssion and Purification of PRL-Analogues

The pET32-a(+) expression vector (Novagen, Madison Wis.) was used for expression of proteins. Recombinant proteins were produced as inclusion bodies in Escherichia coli BL21(DE3) cells co-transfected with pACYCDuet-MetAP plasmid, which express the E. coli MetAP protein. Solubilized in 8M urea, 0.1 M Tris, 2-20 mM DTT, pH 8.5 buffer and following refolding by dilution into a 20 mM Tris, 0.05% Tween 20, pH 8.0. Protein purification was performed using Source30Q ion exchange columns (Amersham Biosciences) followed by a macro-prep Caramic Hydroxyapatite column (BioRad) and a final size-exclusion chromatography on a Sephadex G25 column. PRL receptor was refolded in two dilution steps, first in 0.4M arginine pH 8.5 and then diluted further in 20 mM Tris, 0.05% Tween 20, pH 8.0.

### Biacore Assay

Ser-PRLR (1-210) (25 μg/ml in 10 mM sodium acetate, pH 3.0), was injected into a Biacore 3000 instrument at a flow rate of 5 μl/min and coupled to a CM5 sensor chip by amine coupling chemistry. PRL and PRL analogues (500 nM in buffer; 20 mM Hepes, pH 7.4, containing 0.1 M NaCl, 2 mM CaCl₂ and 0.005% P20) were then injected over the immobilized receptor for 5 minutes at the same flow rate, followed by a 10-min dissociation period during which buffer was injected, to assess receptor binding affinity. Data evaluation was performed in BiaEvaluation 4.1. Regeneration was accomplished with 4.5 M MgCl₂ between runs.

### Receptor Binding Affinity of N56D, G129R-PRL

The PRL receptor binding affinity of G129R-PRL was compared to that of N56D,G129R-PRL (corresponding to the deamidated form of G129R-PRL), using the Biacore-assay. The receptor binding affinities of G129R-PRL and N56D,G129R-PRL were 27 and 313 nM, respectively. Thus, deamidation of Asp-56 markedly reduces the receptor binding affinity

1. An isolated peptide, which peptide is prolactin or a prolactin analogue, and which binds to the prolactin receptor, said peptide having an amino acid mutation, which mutation results in altered secondary structure at a position corresponding to positions 55, 56 or 57 of SEQ ID No. 1, wherein said method comprises mutating one or more amino acids in the segment corresponding to positions 47-57 of SEQ ID No. 1

2. An isolated peptide, which peptide is a prolactin analogue, which binds to the prolactin receptor, said peptide having an amino acid mutation in the position corresponding to position 55, 56 or 57 of SEQ ID No. 1.

3. An isolated peptide, which peptide is a prolactin analogue which binds to the prolactin receptor, said peptide comprising the amino acid sequence of SEQ ID No. 1 having an amino acid mutation in the position corresponding to position 55, 56 or 57 of SEQ ID No. 1.

4. An isolated peptide according to claim 1, wherein the amino acid residue in the position corresponding to position 55 of SEQ ID No. 1 has been substituted with valine, leucine, tryptophan, tyrosine, phenylalanine, proline or threonine.

5. An isolated peptide according to claim 1, wherein the amino acid residue in the position corresponding to position 56 of SEQ ID No. 1 has been substituted with glycine or a glutamine.

6. An isolated peptide according to claim 1, wherein the amino acid residue in the position corresponding to position 57 of SEQ ID No. 1 has been substituted with valine, leucine, isoleucine, tryptophan, tyrosine, phenylalanine, proline or threonine.

7. An isolated peptide according to claim 1, which is an antagonist of the prolactin receptor.

8. An isolated peptide according to claim 1, which is an agonist of the prolactin receptor.

9. An isolated nucleic acid encoding a peptide according to claim 1.

10. A vector comprising a nucleic acid construct according to claim 9.

11. A host cell comprising a nucleic acid construct of claim 9, or a vector of claim 10.

12. An antibody that specifically binds a peptide according to claim 1.

13. An antibody according to claim 12, which antibody does not bind to a peptide comprising the amino acid sequence of SEQ ID No. 1.

14. A method for stabilising prolactin or a prolactin analogue comprising mutating an amino acid in a position resulting in induction of secondary structure at a position corresponding to positions 55, 56 or 57 of SEQ ID No. 1, wherein said method comprises mutating one or more amino acids in the segment corresponding to positions 47-57 of SEQ ID No. 1.

15. A method for stabilising prolactin or a prolactin analogue comprising mutating an amino acid in a position resulting in induction of secondary structure at a position corresponding to positions 55, 56 or 57 of SEQ ID No. 1.

16. A method according to claim 15, wherein the amino acid residue in the position corresponding to position 56 of SEQ ID No. 1 has been substituted with glycine.

17. A method according to claim 15, wherein the amino acid residue in the position corresponding to position 55 of SEQ ID No. 1 has been substituted with valine, leucine, tryptophan, tyrosine, phenylalanine, proline or threonine.

18. A method according to claim 15, wherein the amino acid residue in the position corresponding to position 57 of SEQ ID No. 1 has been substituted with valine, leucine, isoleucine, tryptophan, tyrosine, phenylalanine, proline or threonine.

19. (canceled)

20. (canceled)

21. A pharmaceutical formulation comprising a peptide according to claim 1.

22. (canceled)

23. (canceled)

24. (canceled)

25. (canceled)


SEQUENCE LISTING (1 sequence)

SEQ ID NO: 1 - protein, 199 aa, Homo sapiens
LPICPGGAARCQVTLRDLFDRAVVLSHYIHNLSSEMFSEFDKRYTHGRGFITKAINSCHTSSLATPEDKEQAQQMNQKDFLSLIVSILRSWNEPLYHLVTEVRGMQEAPEAILSKAVEIEEQTKRLLEGMELIVSQVHPETKENEIYPVWSGLPSLQMADEESRLSAYYNLLHCLRRDSHKIDNYLKLLKCRIIHNNNC